(19) United States
(12) Patent Application Publication
(10) Pub. No.: US 2017/0215832 A1
(43) Pub. Date: Aug. 3, 2017

(54) RADIOGRAPHIC IMAGE CAPTURING SYSTEM

(71) Applicant: KONICA MINOLTA, INC., Tokyo (JP)

(72) Inventor: Shigeo NAGANO, Tokyo (JP)

(21) Appl. No.: 15/422,993

(22) Filed: Feb. 2, 2017

(30) Foreign Application Priority Data
Feb. 3, 2016 (JP) 2016-018561

(51) Int. Cl. A61B 6/00

(52) U.S. Cl. CPC A61B 6/56; A61B 6/4405; A61B 6/4233; A61B 6/46; A61B 6/563

(57) ABSTRACT

A radiographic image capturing system includes: a medical cart including a radiation generating apparatus emitting radiation and a first chargeable built-in power supply; a portable radiographic image capturing apparatus including radiation detecting elements and a second chargeable built-in power supply; and a control device managing electric energy remaining in the first built-in power supply and in the second built-in power supply, determines availability of charge of the second built-in power supply in the portable radiographic image capturing apparatus with power from the first built-in power supply in the medical cart based on the electric energy remaining in the first built-in power supply, and performs control so as to allow the charge when the charge is determined to be available, and so as not to conduct the charge when the charge is determined to be unavailable.

### CROSS REFERENCE TO RELATED APPLICATIONS

The present invention claims priority under 35 U.S.C. §119 to Japanese Application No. 2016-018561 filed Feb. 3, 2016, the entire content of which is incorporated herein by reference.

### BACKGROUND OF THE INVENTION

Field of the Invention

The present invention relates to radiographic image capturing systems, in particular, to a radiographic image capturing system equipped with a portable radiographic image capturing apparatus and a medical cart with a radiation generating apparatus mounted thereon.

Description of Related Art

Radiographic image capturing apparatuses (flat panel detectors) have been developed in place of conventional films/screens or photostimulable phosphor plates. For example, there has been developed a radiographic image capturing apparatus equipped with a two-dimensional matrix of radiation detecting elements 7 as shown in FIG. 2 (described below), generating electric charges in accordance with a dose of radiation passing through a subject and received by each radiation detecting element 7, and reading the electric charges as signal values.

Portable radiographic image capturing apparatuses (also called as “FPD cassettes”) have also been developed, each including in a housing a sensor panel with an array of radiation detecting elements disposed thereon (see, for example, FIG. 1, which is described below). The portable radiographic image capturing apparatus is used for simple-image capturing to obtain a single radiographic image by emitting radiation once from the radiation generating apparatus, and for moving-image capturing to obtain plural radiographic images by emitting radiation several times from the radiation generating apparatus.

Since the portable radiographic image capturing apparatus can be carried about, traditional radiation imaging performed in an imaging room in a hospital can be replaced with radiation imaging in a sickroom by bringing the portable radiographic image capturing apparatus and a medical cart on which the radiation generating apparatus mounted into the sickroom and using them there. The portable radiographic image capturing apparatus is hereinafter simply referred to as a radiographic image capturing apparatus.

With reference to FIG. 9, an operator A, such as a radiological technician, moves a radiographic image capturing apparatus 200 and a medical cart 201 to a sickroom SR, insert the radiographic image capturing apparatus 200 between a subject H (patient) and a bed B or apply the radiographic image capturing apparatus 200 to the body of the patient, and then move a medical cart 201 so as to position a radiation generating apparatus 202 above the patient. The operator A causes the radiation generating apparatus 202 to emit radiation X once or several times to perform simple-image or moving-image capturing.

In FIG. 9, the subject H lies on the bed B during imaging. Alternatively, the patient may raise the upper body while lying on bed B, sit on the bed B or a chair (not shown), or stand beside the bed B during imaging.

During the imaging in the sickroom, the radiographic image capturing apparatus and the medical cart may be connected to an outlet in the sickroom with cables to receive power from an external power source. These connections may cause operators, such as radiological technicians, or other patients in the sickroom to stumble over the cables, precluding a proper imaging. To avoid this problem, the radiographic image capturing apparatus and the medical cart each often have its own built-in power supply, which has been charged before use.

Unfortunately, a shortage of electric energy remaining in the built-in power supply in the radiographic image capturing apparatus or in the medical cart precludes simple-image or moving-image capturing in a sickroom to which the radiographic image capturing apparatus and the medical cart are moved.

To solve these problems, Japanese Patent Application Laid-Open Publication No. 2011-110199 notifies an operator, such as radiological technician, of any insufficient charging of the built-in power supply in the radiographic image capturing apparatus, which is charged by supplying power from the built-in power supply in the medical cart to that in the radiographic image capturing apparatus through a connection between them. More specifically, the built-in power supply in the radiographic image capturing apparatus is not charged properly due to a positional deviation of the radiographic image capturing apparatus which is generated during travel on a braille block along a hospital corridor or a step between an elevator and its landing.

Japanese Patent Application Laid-Open Publication No. 2006-141777 discloses a medical cart with a built-in power supply and a built-in electricity-storing capacitor having electric charge therein. When radiation higher than a predetermined level is emitted from the radiation generating apparatus, power is supplied not only from the built-in power supply and but also from the electricity-storing capacitor. This configuration seemingly increases the capacity of the built-in power supply by the power supplied from the electricity-storing capacitor, thus preventing a shortage of power during imaging.

Japanese Patent Application Laid-Open Publication No. 2005-6888 discloses a radiographic image capturing system which issues a warning when the number of images to be captured in the radiographic image capturing apparatus, the number having been specified from an external device, exceeds the number of images that can be captured by the electric energy remaining in the built-in power supply in the radiographic image capturing apparatus.

Unfortunately, radiographic image capturing systems disclosed in the above patent literatures involves separate management of power supply in the radiographic image capturing apparatus and in the medical cart. Such management has the following problems: When the electric energy remaining in the built-in power supply in the radiographic image capturing apparatus is reduced to a predetermined level or lower, the built-in power supply is charged with power from the built-in power supply in the medical cart, which, in turn, results in a reduction in the electric energy remaining in the built-in power supply in the medical cart, precluding necessary cycles of emission of radiation with necessary intensity from the radiation generating apparatus. Accordingly, the electric energy remaining in the built-in power supply in the medical cart cannot be precisely predicted.

In addition, charging the built-in power supply in the radiographic image capturing apparatus results in a reduction in electric energy remaining in the built-in power supply in the medical cart, which may preclude the move of the medical cart to a charging station. This problem also precludes an efficient operation of the entire radiographic image capturing system equipped with the radiographic image capturing apparatus and the medical cart.

Furthermore, such imprecise prediction of the electric energy or an inefficient operation of the radiographic image capturing system may reduce usability for operators, such as radiological technician, or increase burden on patients, who have to wait for a long time. Re-imaging, if necessary, exposes the subject or patient to excess radiation dose.

These issues will be not problematic if the built-in power supply in the medical cart and that in the radiographic image capturing apparatus are each charged to the full capacity, have sufficient electric energy, and require a small number of imaging operations, i.e., a small number of radiation emitting operations or a small number of radiographic images to be captured. However, they are likely to pose a problem if the built-in power supply in the medical cart and that in the radiographic image capturing apparatus cannot be charged adequately in a situation where the medical cart has to be moved to a sickroom as quickly as possible for prompt imaging or for a large number of imaging operations, such as moving-image capturing.

### SUMMARY OF THE INVENTION

An object of the present invention, which has been made to overcome the disadvantages of the conventional techniques described above, is to properly control the charge of the built-in power supply in the portable radiographic image capturing apparatus by supplying power from the built-in power supply in the medical cart equipped with a radiation generating apparatus to provide a radiographic image capturing system that can operate efficiently.

To achieve the above object, a radiographic image capturing system in which one aspect of the present invention is reflected includes: a medical cart which includes a radiation generating apparatus mounted on the medical cart, the radiation generating apparatus emitting radiation, and a first chargeable built-in power supply; a portable radiographic image capturing apparatus which includes a plurality of radiation detecting elements two-dimensionally arranged and a second chargeable built-in power supply; and a control device which manages electric energy remaining in the first built-in power supply in the medical cart and electric energy remaining in the second built-in power supply in the portable radiographic image capturing apparatus, determines availability of charge of the second built-in power supply in the portable radiographic image capturing apparatus with power from the first built-in power supply in the medical cart based on the electric energy remaining in the first built-in power supply, and performs control so as to allow the charge when the charge is determined to be available, and so as not to conduct the charge when the charge is determined to be unavailable.

A radiographic image capturing system in which another aspect of the present invention is reflected includes: a medical cart which includes a radiation generating apparatus mounted on the medical cart, the radiation generating apparatus emitting radiation, and a first chargeable built-in power supply; a portable radiographic image capturing apparatus which includes a plurality of radiation detecting elements two-dimensionally arranged and a second chargeable built-in power supply; and a control device which manages electric energy remaining in the first built-in power supply in the medical cart and electric energy remaining in the second built-in power supply in the portable radiographic image capturing apparatus, determines availability of charge of the second built-in power supply in the portable radiographic image capturing apparatus with power from the first built-in power supply in the medical cart or availability of charge of the first built-in power supply in the medical cart with power from the second built-in power supply in the portable radiographic image capturing apparatus based on the electric energy remaining in the first built-in power supply in the medical cart and the electric energy remaining in the second built-in power supply in the portable radiographic image capturing apparatus, and performs control so as to conduct the former charge when the former charge is determined to be available or so as to conduct the latter charge when the latter charge is determined to be available, and so as not to conduct the former charge when the former charge is determined to be unavailable or so as not to conduct the latter charge when the latter charge is determined to be unavailable.

### BRIEF DESCRIPTION OF THE DRAWINGS

The present invention will become more fully understood from the detailed description given hereinbelow and the appended drawings, and thus are not intended as a definition of the limits of the present invention, and wherein:

FIG. 1 is a perspective view of a radiographic image capturing apparatus;

FIG. 2 is a block diagram illustrating an equivalent circuit of the radiographic image capturing apparatus;

FIG. 3 is a block diagram illustrating an exemplary configuration for charge of a built-in power supply in the radiographic image capturing apparatus;

FIG. 4 is a perspective view of an exemplary configuration of a medical cart;

FIG. 5 illustrates a connector of the radiographic image capturing apparatus and a connector at the end of a cable of the medical cart connected to each other;

FIG. 6 illustrates an exemplary configuration of a box of the medical cart;

FIG. 7 is a block diagram showing a configuration for charge of a radiographic image capturing system according to a first embodiment;

FIG. 8 illustrates three regions indicating the electric energy remaining in the built-in power supply in the medical cart; and

FIG. 9 illustrates the radiographic image capturing apparatus and the medical cart moved to a sickroom for imaging.

### PREFERRED EMBODIMENT OF THE PRESENT INVENTION

With reference to the drawings, a radiographic image capturing system according to embodiments of the present invention will now be described. The portable radiographic image capturing apparatus is also hereinafter simply referred to as a “radiographic image capturing apparatus”.

### [Configuration of Radiographic Image Capturing Apparatus]

A configuration of the radiographic image capturing apparatus used in the radiographic image capturing system according to this embodiment will now be described.

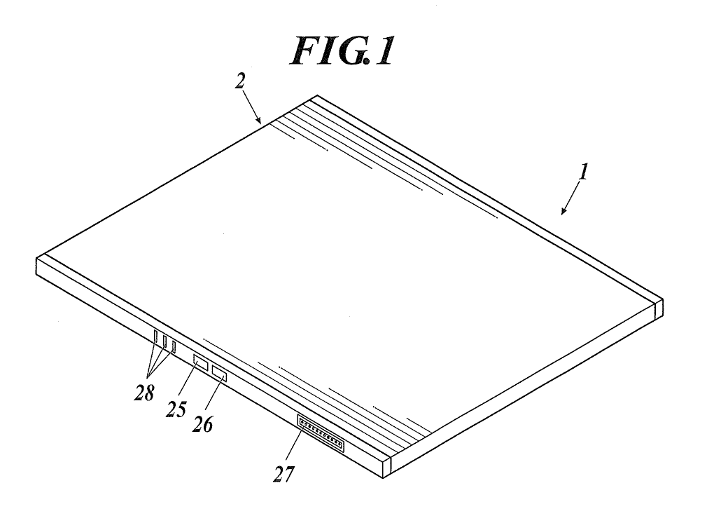

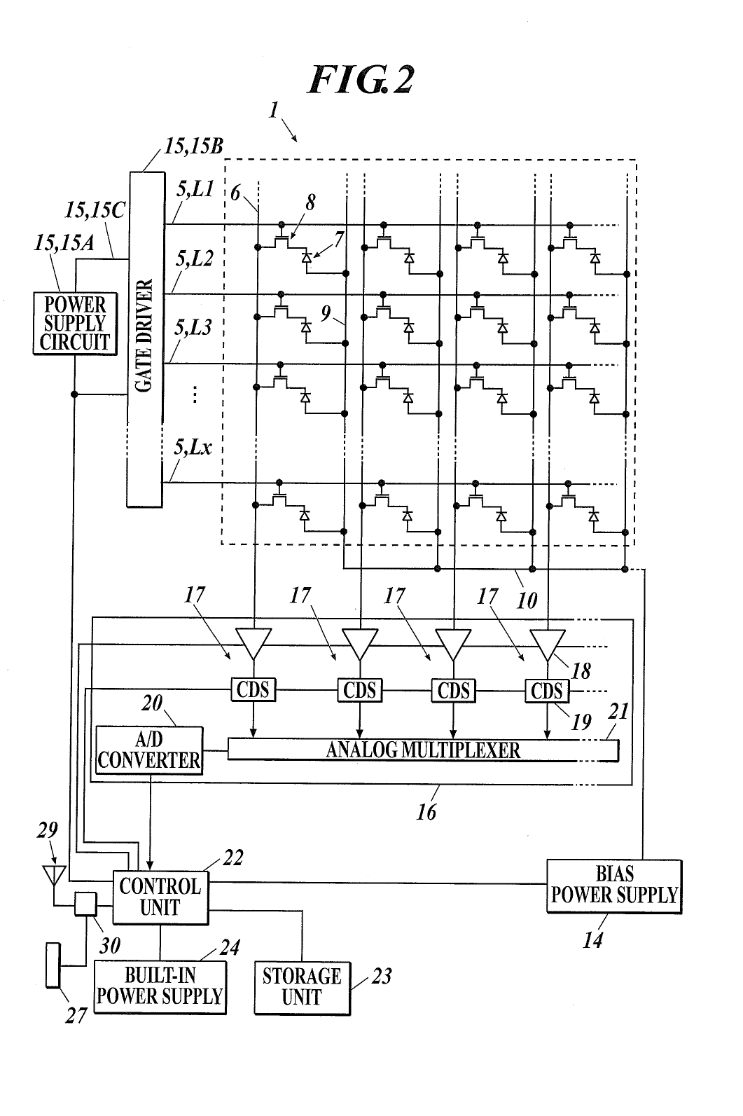

FIG. 1 is a perspective view of the radiographic image capturing apparatus. FIG. 2 is a block diagram illustrating an equivalent circuit of the radiographic image capturing apparatus. As shown in FIGS. 1 and 2, a radiographic image capturing apparatus 1 has a two-dimensional matrix of radiation detecting elements 7 disposed on a sensor substrate (not shown) in a housing 2. As described above, each radiation detecting element 7 generates electric charges in accordance with the dose of radiation exposed to the radiation detecting element 7 through a subject (For a radiographic image capturing apparatus 1 of indirect type, in accordance with the light quantity of electromagnetic waves with a different wavelength into which the incident radiation is converted by a scintillator (not shown) and enters the radiation detecting element 7).

With reference to FIG. 1, the radiographic image capturing apparatus 1 includes a power switch 25, a toggle switch 26, a connector 27 and an indicator 28 disposed on one side of the housing 2. The housing 2 is provided with an antenna 29 (not shown in FIG. 1, see FIG. 2) on, for example, the opposite side of the housing 2 for radio communications with an external device.

Radiation detecting elements 7 are connected to bias lines 9. A reverse bias voltage is applied to the radiation detecting elements 7 via the bias lines 9 and their connection 10 from a bias power supply 14. The radiation detecting elements 7 are also connected to the respective thin film transistors (TFTs) 8, which functions as switching elements. Each TFT 8 is connected to the corresponding signal line 6.

In a scan driving unit 15, on-stage voltage and off-state voltages are applied from a power supply circuit 15A to a gate driver 15B via a line 15C, and switched in the gate driver 15B and applied to scanning lines 5 of L1 to Lx. The off-state voltage applied to each TFT 8 via the corresponding scanning line 5 puts the TFT 8 into the off-state, resulting in disconnection between the corresponding radiation detecting element 7 and the corresponding signal lines 6 to accumulate electric charges in the radiation detecting element 7. In contrast, the on-state voltage applied to each TFT 8 via the corresponding scanning line 5 results in the release of the electric charges accumulated in the corresponding radiation detecting element 7 to the corresponding signal line 6.

Each signal line 6 is connected to the corresponding readout circuit 17 in a readout IC 16. When each radiation detecting element 7 reads a signal value D, the gate driver 15B sequentially applies the on-stage voltage to the scanning lines 5(L1) to 5(Lx). The on-stage voltage applied puts the TFTs 8 into the on-state, which causes the electric charges to flow from the radiation detecting elements 7 into the readout circuits 17 via the TFTs 8 and the signal lines 6. Each amplifying circuit 18 outputs a voltage value in proportion to the electric charges received.

Correlated double sampling circuits 19 (CDSs in FIG. 2) each read the voltage value output from the corresponding amplifying circuit 18 as an analog signal value D and sequentially send the analog signal values D to an A/D converter 20 via an analog multiplexer 21. The analog signal values D are sequentially converted into digital signal values D at the A/D converter 20 and the digital signal values are sequentially stored in a storage unit 23.

A control unit 22 may be a computer provided with a central processing unit (CPU, not shown), a read only memory (ROM), a random access memory (RAM), and an input/output interface, each being connected to a bus. Alternatively, the control unit 22 may be a field programmable gate array (FPGA). Alternatively, the control unit 22 may include a dedicated control circuit.

The control unit 22 is connected to the storage unit 23, a chargeable built-in power supply 24, and a communication unit 30. The storage unit 23 includes a static RAM (SRAM), a synchronous DRAM (SDRAM), or NAND flash memory. The built-in power supply 24 includes a lithium ion capacitor. The communication unit 30 communicates with an external device through a wireless or wired network via the antenna 29 and/or the connector 27.

As described above, the control unit 22 controls the application of reverse bias voltage to each radiation detecting element 7 from the bias power supply 14 and controls the operation of the scan driving unit 15 and the readout circuits 17 to read signal values D from radiation detecting elements 7 and store the read signal values D in the storage unit 23 or transfer them to an external device through the communication unit 30.

The radiographic image capturing apparatus 1 according to this embodiment can operate in a wake-up or imageable mode and a sleep or energy-saving mode. In the wake-up mode, which is also referred to as an imaging or power consumption mode, imaging can be performed while power is supplied to, at least, the control unit 22, the scan driving unit 15, and the readout IC 16 (i.e., the readout circuits 17, etc.). In the sleep mode, power is supplied to only the functional units that are required to receive signals from an external device, such as the communication unit 30, and no power is supplied to the control unit 22 or the readout circuits 17. Even if radiation enters the radiographic image capturing apparatus 1 in the sleep mode, no signal value D is detected and thus no image is captured because the control unit 22 and the readout circuits 17 do not function.

A wake-up signal sent from an external device to the radiographic image capturing apparatus 1 in the sleep mode causes the communication unit 30 to activate or wake up the control unit 22 and any required functional unit.

### [Configuration for Charge of the Built-in Power Supply in the Radiographic Image Capturing Apparatus]

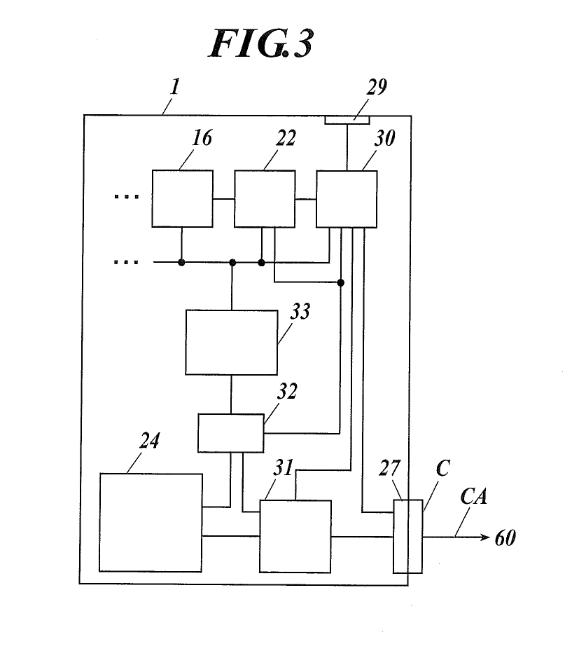

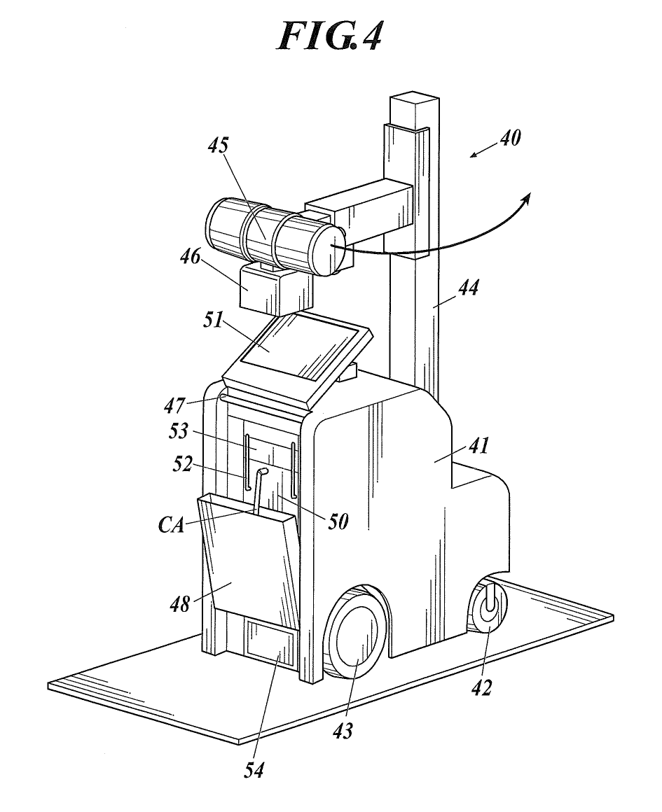

The configuration for charge of the built-in power supply 24 in the radiographic image capturing apparatus 1 according to this embodiment will now be described. FIG. 3 is a block diagram showing an exemplary configuration for charge of the built-in power supply in the radiographic image capturing apparatus. In this embodiment, the connector 27 (see FIG. 1) of the radiographic image capturing apparatus 1 is connected to a connector C at the end of a cable CA (see FIG. 4, described below) extending from a medical cart 40 (described below) or to a connector C of a charging device (not shown) before charge.

With reference to FIG. 3, a charge controlling circuit 31 according to this embodiment, which controls the charge of the built-in power supply 24, is disposed on a charging route between the built-in power supply 24 and the connector 27 in the radiographic image capturing apparatus 1. The charge controlling circuit 31 performs a constant current charge or constant voltage charge based on a voltage V24 of the built-in power supply 24 measured by a voltage measuring unit 32 until the voltage V24 of the built-in power supply 24 reaches a target voltage Vt or the voltage V24 of the built-in power supply 24 increases by a target potential ΔVt.

While the radiographic image capturing apparatus 1 is being in use, the power supply circuit 33 receives power from the built-in power supply 24 and then converts or adjusts its voltage value, as needed, so that the power supplied to each functional unit from the built-in power supply 24 is suitable for the unit. In the sleep mode, power is supplied from the power supply circuit 33 to only the required functional units, such as the communication unit 30, as described above.

### [Medical Cart]

The medical cart included in the radiographic image capturing system according to this embodiment will now be described. FIG. 4 is a perspective view of an exemplary configuration of the medical cart. In the following description, the side provided with an arm 44 of the radiation generating apparatus 45 is defined as the front of the medical cart 40 and the side provided with a handle bar 47 as the rear thereof.

With reference to FIG. 4, the medical cart 40 according to this embodiment is provided with front wheels 42 and the rear wheels 43 in the lower part of the front and the rear of a body 41, respectively. The arm 44 is erected substantially vertically in front of the body 41 of the medical cart 40 and provided with a radiation generating apparatus 45 that can emit radiation.

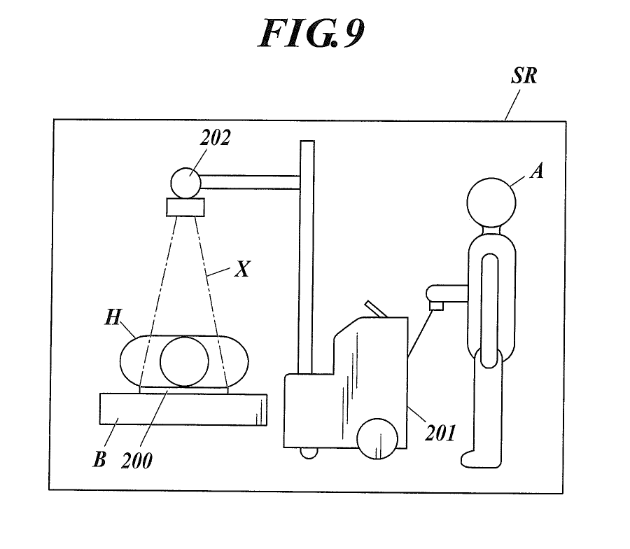

In this embodiment, the arm 44 can be rotated or the radiation generating apparatus 45 can be turned about the arm 44. During the move of the medical cart 40, the radiation generating apparatus 45 is positioned as shown in FIG. 4. During imaging (see FIG. 9), the radiation generating apparatus 45 is turned in the direction of the arrow from the position shown in FIG. 4. The radiation generating apparatus 45 is provided with a collimator 46 used to limit the irradiation field of emitted radiation.

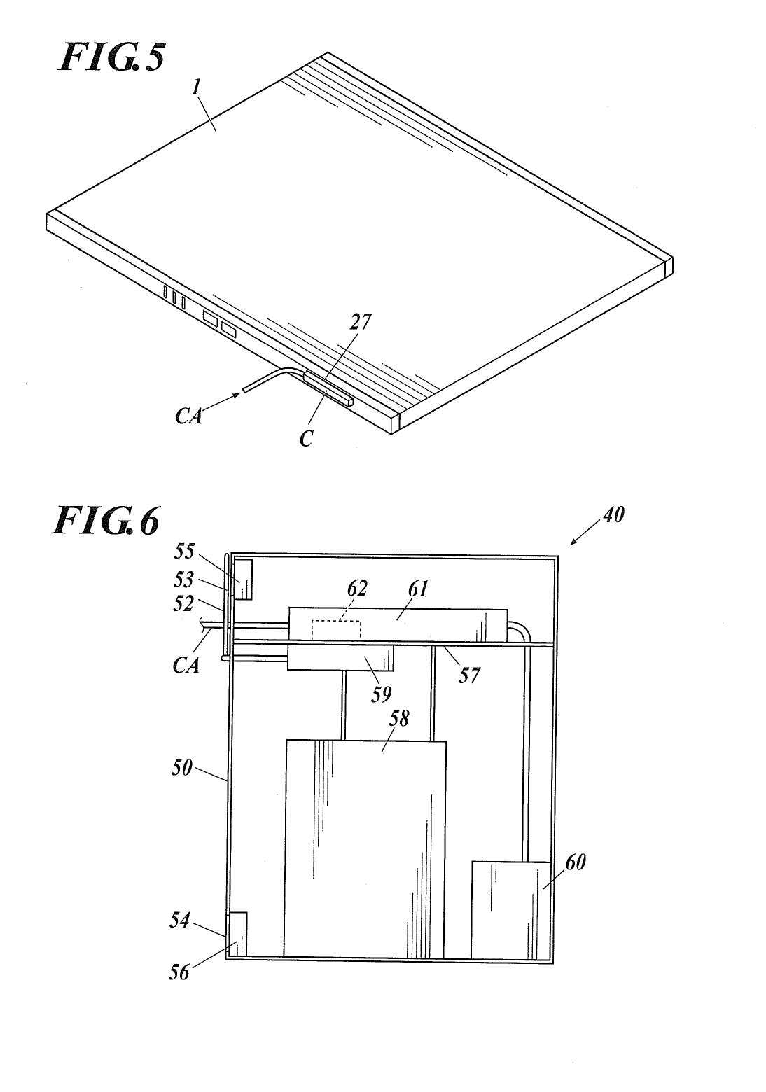

The medical cart 40 according to this embodiment is provided with a display screen 51 above the top of the body 41 of the medical cart 40. The display screen 51 is connected to a computer unit 58 (see FIG. 6, described below) accommodated in a box 50 (described below). The display screen 51 according to this embodiment is a touch panel which also functions as an input device of the computer unit 58. The display screen 51 may be provided with an input unit, such as a mouse or a keyboard.

The body 41 of the medical cart 40 is provided with a handle bar 47 at upper rear. The handle bar 47 is gripped by an operator, such as a radiological technician, to move the medical cart 40. The body 41 of the medical cart 40 is provided with a cassette holder 48 holding the radiographic image capturing apparatus 1 (see FIG. 1) on the rear thereof. This configuration allows the radiographic image capturing apparatus 1, which is used for imaging, to be transported together with the medical cart 40 just by putting it in the cassette holder 48.

The cassette holder 48 according to this embodiment accommodates the cable CA and the connector C (not shown in FIG. 4, see FIGS. 3 and 5, described below) attached at its end. The cable CA extends from an interface unit 61 in the box 50 to the exterior of the box 50.

The radiographic image capturing apparatus 1 is inserted into the cassette holder 48 of the medical cart 40 while the connector C is connected to the connector 27 of the radiographic image capturing apparatus 1 such that the radiographic image capturing apparatus 1 is connected to the interface unit 61 via the cable CA, as shown in FIG. 5.

As shown in FIG. 4, an antenna 52 of an access point 59 (described below) in the box 50 is exposed from the rear of the box 50. The box 50 is provided with an inlet 53 at the upper rear and with an outlet 54 at the lower rear.

The box 50 is accommodated in the body 41 of the medical cart 40. FIG. 6 illustrates an exemplary configuration of the box 50. To facilitate understanding, the box 50 is detached from the body 41 of the medical cart 40 and one side plate is removed to show its content. As described above, the inlet 53 is provided at the upper rear of the box 50 and an anti-dust filter 55 is provided inside the box 50 adjacent to the inlet 53. The outlet 54 is provided at the lower rear of the box 50 and an exhaust fan 56 is provided inside the box 50 adjacent to the outlet 54.

The box 50 is provided with a partition plate 57 that separates the inside of the box into an upper space and a lower space. The lower space in the box 50 is provide with a computer unit 58, which controls imaging, the access point 59 (described above) and a built-in power supply 60 for the medical cart 40. The built-in power supply 60 in the medical cart 40 according to this embodiment includes a chargeable battery or secondary cell.

The upper space in the box 50 is provided with an interface unit 61 and a charging unit 62. Furthermore, the box 50 is provided with various devices (not shown), such as a generator (control section) of the radiation generating apparatus 45 and a motor that drives the rear wheels 43 of the medical cart 40.

The computer unit 58 according to this embodiment is a general-purpose computer that includes a not-shown CPU, ROM, RAM and input/output interface, each being connected to a bus. Alternatively, the computer unit 58 may be a dedicated computer. The computer unit 58 is connected to the display screen 51 (see FIG. 4) via the interface unit 61, although not shown in FIG. 6.

The computer unit 58 according to this embodiment functions as a control device of the present invention. Hence, the computer unit 58 functioning as the control device of the present invention is hereinafter referred to as the “control device 58”. Alternatively, the control device of the present invention may be any device, which will be described below, other than the computer unit 58 in the medical cart 40.

The interface unit 61 relays transmission/reception of signals and data between different devices. The charging unit 62 feeds power from an external power supply to the built-in power supply 60 in the medical cart 40 to charge the built-in power supply 60 and has a function equivalent to the charge controlling circuit 31 (see FIG. 3) of the radiographic image capturing apparatus 1. The charging unit 62 according to this embodiment also has a function to adjust power or voltage when the built-in power supply 24 in the radiographic image capturing apparatus 1 (see FIGS. 2 and 3) is charged with power fed from the built-in power supply 60 in the medical cart 40.

The charging unit 62 measures the voltage V60 of the built-in power supply 60 in the medical cart 40 during charge, and thus functions as a voltage measuring unit. With reference to FIG. 6, the charging unit 62 is accommodated in the interface unit 61. Alternatively, the charging unit 62 may be separated from the interface unit 61.

### [Configuration Specific to the Present Invention]

A configuration specific to the radiographic image capturing system according to this embodiment of the present invention and its operations will now be described.

The radiographic image capturing system according to the present invention comprehensively manages the electric energy E24 remaining in the built-in power supply 24 in the radiographic image capturing apparatus 1 and the electric energy E60 remaining in the built-in power supply 60 in the medical cart 40 to efficiently operate the radiographic image capturing system, which is provided with the radiographic image capturing apparatus 1 and the medical cart 40.

As described above, the capacity or chargeable electric energy of the built-in power supply 24 in the radiographic image capturing apparatus 1 (see FIGS. 2 and 3) is incommensurably lower than that of the built-in power supply 60 in the medical cart 40 (see FIG. 6). Thus, it is unlikely to charge the built-in power supply 60 in the medical cart 40 with power from the built-in power supply 24 in the radiographic image capturing apparatus 1. The following description begins with charge of the built-in power supply 24 in the radiographic image capturing apparatus 1 with power from the built-in power supply 60 in the medical cart 40.

During the move of the medical cart 40, the radiographic image capturing apparatus 1 is in the cassette holder 48 of the medical cart 40 (see FIG. 4) and the connector 27 of the radiographic image capturing apparatus 1 is connected to the connector C of the cable CA of the medical cart 40 (see FIG. 5). At this time, the control device 58 can automatically charges the built-in power supply 24 in the radiographic image capturing apparatus 1 without the intervention of the operator A, such as a radiological technician. The description of the first embodiment assumes that the control device 58 automatically charges the built-in power supply 24 in the radiographic image capturing apparatus 1 while the connector 27 and the connector C are connected to each other.

While the connector 27 of the radiographic image capturing apparatus 1 is being disconnected from the connector C of the cable CA of the medical cart 40, the control device 58 cannot charge the built-in power supply 24 in the radiographic image capturing apparatus 1 by automatic power feed from the built-in power supply 60 in the medical cart 40. The description of the second embodiment will focuses on how the control device 58 controls charge during disconnection of the connector 27 from the connector C.

At present, an attempt is made to increase the capacity of the built-in power supply 24 in the radiographic image capturing apparatus 1. If the capacity of the built-in power supply 24 in the radiographic image capturing apparatus 1 becomes larger in future, the built-in power supply 60 in the medical cart 40 could be charged with power from the built-in power supply 24 in the radiographic image capturing apparatus 1.

Furthermore, the built-in power supply 60 in the medical cart 40 and the built-in power supply 24 in the radiographic image capturing apparatus 1 could charge each other by exchanging power between them. The description of the third embodiment will focus on charging the built-in power supply 60 in the medical cart 40 with power from the built-in power supply 24 in the radiographic image capturing apparatus 1.

### First Embodiment

A radiographic image capturing system 100 according to the first embodiment of the present invention will now be described. The control device 58 manages the electric energy E60 remaining in the built-in power supply 60 in the medical cart 40 and the electric energy E24 remaining in the built-in power supply 24 in the radiographic image capturing apparatus 1 and determines the availability of charge of the built-in power supply 24 in the radiographic image capturing apparatus 1 with power from the built-in power supply 60 in the medical cart 40 based on these remaining levels of electric energy E60, E24. If the charge is determined to be available, charge is conducted under instruction of the control device 58; otherwise, no charge is conducted. Details will now be described below.

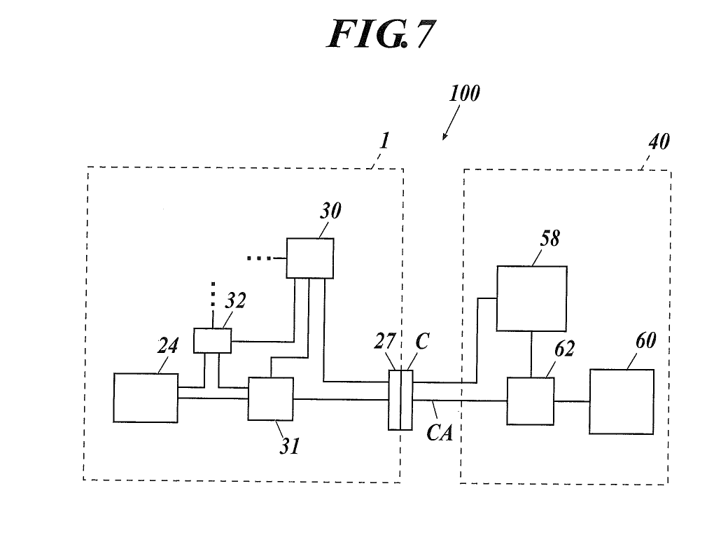

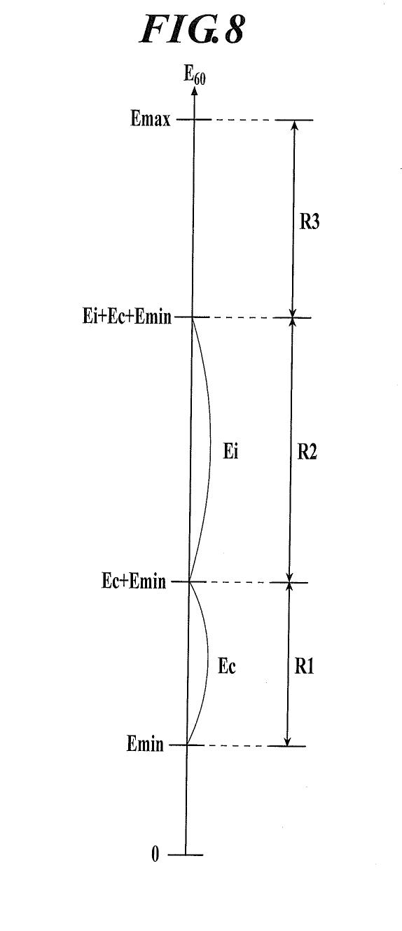

FIG. 7 is a block diagram showing a configuration for charge of the radiographic image capturing system 100 according to the first embodiment. In this embodiment, the connector 27 of the radiographic image capturing apparatus 1 is connected to the connector C of the medical cart 40 and the control device 58 instructs automatic charge of the built-in power supply 24 in the radiographic image capturing apparatus 1, as described above. At the start of charge, for example, the control device 58 may send a message to the display screen 51 (see FIG. 4) to notify the operator A, such as a radiological technician, of the start and wait for the operator A to approve the charge.

[Classification of Electric Energy Remaining in the Built-in Power Supply in the Medical Cart into Regions]

The control device 58 according to this embodiment classifies the electric energy E60 remaining in the built-in power supply 60 in the medical cart 40 into the following three regions before the start of imaging:
(A) First region R1, substantially no electric energy E60 remains in the built-in power supply 60 in the medical cart 40;
(B) Second region R2, not much electric energy E60 remains in the built-in power supply 60 in the medical cart 40; and
(C) Third region R3, enough electric energy E60 remains in the built-in power supply 60 in the medical cart 40

The electric energy E60 remaining in the built-in power supply 60 in the medical cart 40 will now be described. With reference to FIG. 8, the electric energy E60 remaining in the built-in power supply 60 normally has an upper limit Emax and a lower limit Emin. (These limits may be voltage values.)

The medical cart 40 is very heavy due to the radiation generating apparatus 45, the motor, and the built-in power supply 60 included therein. If the medical cart 40 no longer moves due to running out of battery, moving it to a charging station within the hospital would require a lot of work, which significantly reduces the efficiency of imaging.

When a sickroom SR for imaging is determined, the control device 58 calculates the electric energy required to move the medical cart 40 from the current position (for example, storage of the medical cart 40, which may also function as a charging station) to the sickroom SR and then to the charging station. The control device 58 determines the calculated electric energy to be electric energy Ec required to move the medical cart 40 (herein after referred to as the “electric energy Ec required for move”) and positions the electric energy Ec above, in terms of energy, the lower limit Emin of the electric energy E60, as shown in FIG. 8.

The electric energy Ec required for move may be determined as follows: For example, the electric energy required to move the medical cart 40 from each sickroom SR to the charging station is summarized into a table. When a sickroom SR for imaging is specified, the control device 58 determines the electric energy Ec required for move of the medical cart 40 with reference to the table. Alternatively, the maximum electric energy Ec required for move may be preliminarily selected as a fixed value, although the electric energy required to move the medical cart 40 from each sickroom SR in the hospital to the charging station varies, depending on its position.

As shown in FIG. 8, the control device 58 determines the first region R1 from the lower limit Emin of the built-in power supply 60 in the medical cart 40 to a total (Ec+Emin) of the power (Ec) required for move and the lower limit Emin to be the region (A) having substantially no electric energy E60 remaining in the built-in power supply 60 in the medical cart 40.

Information on imaging performed in the sickroom SR to which the medical cart 40 is to be moved, that is, imaging order information is entered in the computer unit 58 in the medical cart 40 in advance, i.e., before the move of the medical cart 40. Alternatively, such necessary imaging order information may be automatically received via the computer unit 58 in the medical cart 40 or a console (now shown) from a hospital information system (HIS) (not shown) or a radiology information system (RIS) in response to an operation by the operator A, such as a radiological technician.

The imaging order information, which contains information on a portion to be imaged and simple-image or moving-image capturing operations, allows the control device 58 to calculate electric energy required to emit radiation from the radiation generating apparatus 45 for imaging (for simple-image capturing, power required for a single emission and for moving-image capturing, electric energy required for multiple emissions) based on, for example, the entered imaging order information.

The control device 58 according to this embodiment adds these levels of electric energy to determine the electric energy Ei required for the radiation generating apparatus 45 to emit radiation for imaging to be performed (hereinafter referred to as the “electric energy Ei required for radiation emission”). As shown in FIG. 8, the control device 58 positions the electric energy Ei required for radiation emission above, in terms of energy, the electric energy Ec required for move.

As shown in FIG. 8, the control device 58 determines the second region R2 from the total (Ec+Emin) to a total (Ei+Ec+Emin required for radiation emission) to be the region (B) having not much electric energy E60 remaining in the built-in power supply 60 in the medical cart 40.

The control device 58 determines the third region R3 above the total (Ei+Ec+Emin) to be the region (C) having enough electric energy E60 remaining in the built-in power supply 60 in the medical cart 40.

As described below, the control device 58 switches the charge control, depending on the regions (first region R1, second region R2, or third region R3 shown in FIG. 8) to which the electric energy E60 remaining in the built-in power supply 60 in the medical cart 40 belongs when the built-in power supply 24 in the radiographic image capturing apparatus 1 is to be charged with power from the built-in power supply 60 in the medical cart 40.

### [Calculation of Electric Energy Actually Remaining in the Built-in Power Supply in the Medical Cart]

The control device 58 according to this embodiment calculates the electric energy E60 actually remaining in the built-in power supply 60 in the medical cart 40 based on the voltage V60 of the built-in power supply 60 in the medical cart 40 measured by the charging unit 62 of the medical cart 40 at the time when the connector C of the medical cart 40 is connected to the connector 27 of the radiographic image capturing apparatus 1 before imaging starts.

If the electric energy E60 remaining in the built-in power supply 60 in the medical cart 40 is already equal to or less than the total (Ec+Emin) of the lower limit Emin of the built-in power supply 60 in the medical cart 40 and the electric energy Ec required for move (see FIG. 8), the control device 58 displays a message on, for example, the display screen 51 of the medical cart 40 (see FIG. 4) or raises alarm to direct the operator A, such as a radiological technician, to move the medical cart 40 to the charging station to charge the built-in power supply 60.

### [Acquisition of Information on Voltage V24 of the Built-in Power Supply in the Radiographic Image Capturing Apparatus]

After determining the first to third regions R1 to R3 as described above, the control device 58 determines the necessity for charge of the built-in power supply 24 in the radiographic image capturing apparatus 1. The control device 58 thus acquires information on the voltage V24 of the built-in power supply 24 in the radiographic image capturing apparatus 1 measured by the voltage measuring unit 32 of the radiographic image capturing apparatus 1.

The radiographic image capturing apparatus 1 is placed in the cassette holder 48 of the medical cart 40 (see FIG. 4) while the connector 27 is connected to the connector C of the medical cart 40, as shown in FIG. 5. At that time, the power consumption mode of the radiographic image capturing apparatus 1 is set to the sleep mode to avoid a wasteful consumption of power in many cases.

The control device 58 sends a signal requesting to send voltage V24 to the radiographic image capturing apparatus 1. The communication unit 30 of the radiographic image capturing apparatus 1 receives the signal, activates the voltage measuring unit 32 of the radiographic image capturing apparatus 1 to measure the voltage V24 of the built-in power supply 24, obtains the measured voltage V24, and then sends it to the control device 58. If the imaging mode of the radiographic image capturing apparatus 1 is set to the wake-up mode, the sending request signal is sent from the communication unit 30 to the control unit 22, which performs the above process.

### [Determination of the Availability of Charge of the Built-in Power Supply in the Radiographic Image Capturing Apparatus—Part 1]

Upon obtaining the information on the voltage V24 of the built-in power supply 24 in the radiographic image capturing apparatus 1, the control device 58 calculates the electric energy E24 remaining in the built-in power supply 24 in the radiographic image capturing apparatus 1 based on the obtained voltage V24. Alternatively, a corresponding table between the voltage V24 of the built-in power supply 24 in the radiographic image capturing apparatus 1 and the electric energy E24 remaining in the built-in power supply 24 may be created preliminarily so that the control device 58 can determine the electric energy E24 remaining in the built-in power supply 24 in the radiographic image capturing apparatus 1 with reference to the table.

The control device 58 determines the availability for the radiographic image capturing apparatus 1 to perform each imaging designated in the imaging order information. In other words, the control device 58 determines whether the electric energy required for proper read operations of the signal values D for each imaging remains in the built-in power supply 24 in the radiographic image capturing apparatus 1 before imaging.

More specifically, the control device 58 calculates the total electric energy, consumed in reading of the signal values D in the radiographic image capturing apparatus 1, of the built-in power supply 24 in the radiographic image capturing apparatus 1 (hereinafter referred to as the “electric energy Ef required for imaging”) for one or more imaging operations designated in the imaging order information, and determines whether the electric energy E24 remaining in the built-in power supply 24 in the radiographic image capturing apparatus 1 is equal to or more than the electric energy Ef required for the imaging.

If the control device 58 determines that the electric energy E24 remaining in the built-in power supply 24 in the radiographic image capturing apparatus 1 is equal to or greater than the electric energy Ef required for imaging, the control device 58 determines that enough electric energy E24 remains in the built-in power supply 24 in the radiographic image capturing apparatus 1 and that no charge is necessary for the built-in power supply 24 and does not direct to perform charge, in other words, to feed power to the built-in power supply 24 in the radiographic image capturing apparatus 1 from the built-in power supply 60 in the medical cart 40.

[On Transfer of Signal Values from the Radiographic Image Capturing Apparatus]

When the medical cart 40 is moved to a sickroom SR, the radiographic image capturing apparatus 1 is detached from the cassette holder 48 of the medical cart 40 and is placed between the subject H (patient) and the bed B, as shown in FIG. 9 before imaging. Once imaging is performed, the radiographic image capturing apparatus 1 reads signal values D, as described above.

The read signal values D are temporarily stored in the storage unit 23 (see FIG. 2) and transferred to the computer unit 58 (see FIG. 6) in the medical cart 40 or an external device, such as console (not shown). The radiographic image capturing apparatus 1 and the medical cart 40 may be connected via the cable CA (see FIGS. 4 and 5) to transfer the signal values D through a wired network.

In this case, the cable CA may cause the operator A or other patients to stumble over the cable, resulting in the movement of the radiographic image capturing apparatus 1 by the stretched cable CA. If the medical cart 40 is moved to a sickroom SR for imaging, the signal values D read by the radiographic image capturing apparatus 1 are transferred through a wireless network via the communication unit 30 and the antenna 29 in many cases (see FIG. 2).

In this configuration, which allows information to be transferred through a wireless network, the electric energy E24 remaining in the built-in power supply 24 in the radiographic image capturing apparatus 1 must be equal to or greater than a total (Ef+Ew1) of the electric energy Ef required to read the signal values D and the electric energy Ew1 required to transfer the signal values D via a wireless network. The electric energy Ew1 required for transfer cannot be replenished because the cable CA is not connected to the radiographic image capturing apparatus 1 during imaging.

The control device 58 calculates the electric energy E24 remaining in the built-in power supply 24 in the radiographic image capturing apparatus 1, as described above. If the electric energy E24 remaining in the built-in power supply 24 in the radiographic image capturing apparatus 1 is equal to or greater than the electric energy Ef required for imaging, the control device 58 determines that the built-in power supply 24 is not to be charged. In this case, if the electric energy E24 is equal to or greater than a total (Ef+Ew1) of the electric energy required to read the signal values D (i.e., the electric energy Ef required for imaging) and the electric energy Ew1 required to transfer the signal values D via a wireless network, the control device 58 instructs the radiographic image capturing apparatus 1 to transfer the read signal values D to an external device through the wireless network for each imaging.

If the electric energy E24 remaining in the built-in power supply 24 in the radiographic image capturing apparatus 1 is less than a total (Ef+Ew1) of the electric energy required to read the signal values D (i.e., the electric energy Ef required for imaging) and the electric energy Ew1 required to transfer the signal values D via the wireless network, the control device 58 may direct the radiographic image capturing apparatus 1 to store the read signal values D in the storage unit 23. In this case, the radiographic image capturing apparatus 1 is connected to the computer unit 58 in the medical cart 40 or the console via the cable CA to transfer the signal values D through the wired network after the completion of all the imaging operations designated in the imaging order information.

This configuration is useful to prevent the following risk: The transfer of the signal values D from the radiographic image capturing apparatus 1 through the wireless network for each imaging while the electric energy E24 remaining in the built-in power supply 24 in the radiographic image capturing apparatus 1 is not enough results in the depletion of the electric energy E24 remaining in the built-in power supply 24 in the radiographic image capturing apparatus 1 (running out of battery) before all the imaging operations designated in the imaging order information are completed. Accordingly, this configuration can ensure the continuation of imaging.

For moving-image capturing, in particular, running out of battery in the middle of repeated imaging results in having to resume moving-image capturing over again from the beginning (re-imaging), which leads to an increased radiation dose of the patient. Advantageously, the above configuration can successfully avoid such a problem.

If enough electric energy E60 remains in the built-in power supply 60 in the medical cart 40, at least the electric energy Ew1 required to transfer the signal values D via the wireless network may be preliminarily charged from the built-in power supply 60 in the medical cart 40 to the built-in power supply 24 in the radiographic image capturing apparatus 1.

### [Determination of the Availability of Charge of the Built-in Power Supply in the Radiographic Image Capturing Apparatus—Part 2]

The control device 58 calculates the electric energy E24 remaining in the built-in power supply 24 in the radiographic image capturing apparatus 1 based on information on the voltage V24 of the built-in power supply 24 obtained from the radiographic image capturing apparatus 1, as shown above. If the remaining electric energy E24 is equal to or less than the electric energy Ef required for imaging in the radiographic image capturing apparatus 1, the control device 58 determines that the electric energy E24 remaining in the built-in power supply 24 in the radiographic image capturing apparatus 1 is not enough and then determines the availability of charge of the built-in power supply 24 in the radiographic image capturing apparatus 1 with power from the built-in power supply 60 in the medical cart 40 based on the electric energy E60 remaining in the built-in power supply 60 in the medical cart 40.

The charge of the built-in power supply 24 in the radiographic image capturing apparatus 1 with power from the built-in power supply 60 in the medical cart 40 reduces the electric energy E60 remaining in the built-in power supply 60 in the medical cart 40. The control device 58 determines one of regions R1 to R3 in FIG. 8 to which the reduced electric energy E60 remaining in the built-in power supply 60 in the medical cart 40 belongs.

### [Calculation of Electric Energy Supplied to the Built-in Power Supply in the Radiographic Image Capturing Apparatus]

The control device 58 calculates electric energy required to be fed from the built-in power supply 60 in the medical cart 40 to the built-in power supply 24 in the radiographic image capturing apparatus 1, that is a difference ΔE by subtracting the electric energy E24 remaining in the built-in power supply 24 in the radiographic image capturing apparatus 1, calculated as shown above, from the electric energy Ef required for imaging (or the total (Ef+Ew1) of the electric energy Ef required for imaging and the electric energy Ew1 required to transfer the signal values D via the wireless network, the same applies hereinafter).

In the following treatment, a sum of a predetermined electric energy and the difference ΔE may be used instead of the difference ΔE, to allow a bit of leeway for the electric energy E24 remaining in the built-in power supply 24 in the radiographic image capturing apparatus 1.

### [Electric Energy Belonging to the First Region R1—No Charge]

The control device 58 determines one of regions (R1 to R3) to which the electric energy E60 remaining in the built-in power supply 60 in the medical cart 40 after the charge of the built-in power supply 24 in the radiographic image capturing apparatus 1 with the electric energy equivalent to the difference ΔE (i.e., the electric energy E60 obtained by subtracting the difference ΔE from the electric energy E60 remaining in the built-in power supply 60 in the medical cart 40, calculated as shown above, hereinafter referred to as the “post-discharge electric energy E60”) belongs. If the post-discharge electric energy E60 belongs to the first region R1, in other words, the electric energy E60 remaining in the built-in power supply 60 in the medical cart 40 is reduced to a level equal to or less than the total (Ec+Emin), which is the sum of the lower limit Emin of the electric energy of the built-in power supply 60 and the electric energy Ec required to move the medical cart, the control device 58 determines the charge of the built-in power supply 24 in the radiographic image capturing apparatus 1 to be unavailable and does not instruct charge.

If the post-discharge electric energy E60 is greater than the total (Ec+Emin), but a difference between the post-discharge electric energy E60 and the total (Ec+Emin) is so small as to preclude a single radiation operation from the radiation generating apparatus 45, the medical cart 40 moved to a sickroom SR should be moved to the charging station with no radiation operation.

To avoid such a risk, the control device 58 determines the charge of the built-in power supply 24 in the radiographic image capturing apparatus 1 to be unavailable without an instruction of charge if the post-discharge electric energy E60 after charging the built-in power supply 24 in the radiographic image capturing apparatus 1 is reduced to a level equal to or less than the total (Ec+Emin+Ea), which is the sum of a predetermined electric energy Ea (for example, electric energy required for a single radiation operation from the radiation generating apparatus 45) and the total (Ec+Emin).

If the charge of the built-in power supply 24 in the radiographic image capturing apparatus 1 from the built-in power supply 60 in the medical cart 40 precludes the move of the medical cart 40 to a sickroom SR and then to the charging station (or results in a transfer of the medical cart 40 from the sickroom SR to the charging station with no radiation operation), the control device 58 does not instruct to charge the built-in power supply 24 in the radiographic image capturing apparatus 1 with power from the built-in power supply 60 in the medical cart 40.

This configuration allows the control device 58 to move the medical cart 40 to the charging station and properly charge the built-in power supply 60 in the medical cart 40 (and the built-in power supply 24 in the radiographic image capturing apparatus 1).

As described above, for example, the move of the medical cart 40 to a sickroom SR with an inadequate electric energy E60 remaining in the built-in power supply 60 in the medical cart 40 may result in transfer of the medical cart 40 to the charging station without capturing any image, charge at the charging station, and then re-transfer to the sickroom SR for imaging. The radiographic image capturing system 100 according to this embodiment allows the medical cart 40 to be moved to a sickroom SR after charging it to enable a smooth imaging and an efficient operation of the radiographic image capturing system 100.

If the built-in power supply 24 in the radiographic image capturing apparatus 1 is not to be charged with power from the built-in power supply 60 in the medical cart 40, the control device 58 preferably displays a message on, for example, the display screen 51 of the medical cart 40 (see FIG. 4) or raise alarm to indicate the inability of imaging or the necessity for charge of the built-in power supply 60 in the medical cart 40.

This configuration allows the operator A, such as a radiological technician, to recognize the necessity for charge of the built-in power supply 60 in the medical cart 40 and move the medical cart 40 to the charging station to charge the built-in power supply 60 in the medical cart 40 (and the built-in power supply 24 in the radiographic image capturing apparatus 1) before moving it to a sickroom SR.

### [In the Case of Charge]

If the post-discharge electric energy E60 after charge of the built-in power supply 24 in the radiographic image capturing apparatus 1 belongs to the second region R2 or R3 (see FIG. 8), the control device 58 determines that the built-in power supply 24 in the radiographic image capturing apparatus 1 is chargeable with power from the built-in power supply 60 in the medical cart 40 and instructs charge.

### [Electric Energy Belonging to Third Region R3]

If the post-discharge electric energy E60 belongs to the third region R3, the built-in power supply 60 in the medical cart 40 has remaining electric energy equal to or greater than the electric energy required for imaging even after charge of the built-in power supply 24 in the radiographic image capturing apparatus 1. As soon as the control device 58 determines to perform charge as shown above, the control device 58 automatically starts the charge of the built-in power supply 24 in the radiographic image capturing apparatus 1 with power from the built-in power supply 60 in the medical cart 40.

At the start of charge, the control device 58 may send a message to the display screen 51 to notify the operator A, such as a radiological technician, of the start and wait for the operator A to approve the charge.

This configuration allows the control device 58 to comprehensively manage the electric energy E60 remaining in the built-in power supply 60 in the medical cart 40 and the electric energy E24 remaining in the built-in power supply 24 in the radiographic image capturing apparatus 1 to charge the built-in power supply 24 in the radiographic image capturing apparatus 1 properly. This configuration also eliminate the necessity for move of the radiographic image capturing apparatus 1 to the charging station for charging and allows the built-in power supply 24 in the radiographic image capturing apparatus 1 to be charged properly with power from the built-in power supply 60 in the medical cart 40, thus enabling an efficient operation of the radiographic image capturing system 100.

### [Electric Energy Belonging to Second Region R2—Part 1]

If the post-discharge electric energy E60 belongs to the second region R2, the radiographic image capturing apparatus 1 can perform each imaging operation designated in the imaging order information after charge, in other words, the signal values D can be read properly in each imaging operation. In contrast, the medical cart 40, which has fed power to the radiographic image capturing apparatus 1, does not have enough post-discharge electric energy E60 (less than (Ei+Ec+Emin)) to perform all the imaging operations designated in the imaging order information, in other words, the medical cart 40 cannot perform all the imaging operations designated in the imaging order information (i.e. the radiation generating apparatus 45 cannot perform a necessary number of radiation emission operations).

In this case, the control device 58, for example, may display a message on, for example, the display screen 51 (see FIG. 4) of the medical cart 40 or raise alarm to instruct the operator A, such as a radiological technician, to move the medical cart 40 to the charging station for charge of the built-in power supply 60.

### [Electric Energy Belonging to Second Region R2—Part 2]

For example, in an emergency imaging, the medical cart 40 may have to be directly moved to a sickroom SR for imaging without moving the medical cart 40 to the charging station or charging the built-in power supply 60. In this case, imaging operations must be performed with the electric energy E60 remaining in the built-in power supply 60 in the medical cart 40 and the electric energy E24 remaining in the built-in power supply 24 in the radiographic image capturing apparatus 1 since there is no time to charge the built-in power supply 60 in the medical cart 40.

If the post-discharge electric energy E60 belongs to the second region R2 in FIG. 8, which precludes the capturing of a necessary number of radiographic images, radiographic images are captured as many as possible with the electric energy E60 remaining in the built-in power supply 60 in the medical cart 40 and the electric energy E24 remaining in the built-in power supply 24 in the radiographic image capturing apparatus 1.

If the built-in power supply 24 in the radiographic image capturing apparatus 1 is not to be charged, imaging can be performed a smaller number between the number of radiographic images that can be captured with the electric energy E24 remaining in the built-in power supply 24 in the radiographic image capturing apparatus 1 (hereinafter referred to as “the number of capturable images N1”) or the number of radiation emission operations from the radiation generating apparatus 45 possible with the electric energy E60 remaining in the built-in power supply 60 in the medical cart 40 (hereinafter referred to as “the possible number of times of radiation emission”).

In this embodiment, the capacity of the built-in power supply 24 in the radiographic image capturing apparatus 1 is incommensurably lower than that of the built-in power supply 60 in the medical cart 40, as described above. Thus, it is most likely that power is fed from the built-in power supply 60 in the medical cart 40 to charge the built-in power supply 24 in the radiographic image capturing apparatus 1, but the opposite is unlikely, i.e., power is fed from the built-in power supply 24 in the radiographic image capturing apparatus 1 to charge the built-in power supply 60 in the medical cart 40.

Accordingly, if the number of capturable images N1 capable of being captured with the electric energy E24 remaining in the built-in power supply 24 in the radiographic image capturing apparatus 1 is greater than the possible number of times of radiation emission N45 from the radiation generating apparatus 45 with the electric energy E60 remaining in the built-in power supply 60 in the medical cart 40, the built-in power supply 24 in the radiographic image capturing apparatus 1 is not to be charged and imaging is performed only the possible number of times of radiation emission N45.

In contrast, if the possible number of times of radiation emission N45 from the medical cart 40 (or the radiation generating apparatus 45) is greater than the number of capturable images N1 in the radiographic image capturing apparatus 1, the built-in power supply 24 in the radiographic image capturing apparatus 1 is charged with power from the built-in power supply 60 in the medical cart 40 to charge.

Charge performed such that the number of capturable images N1 in the radiographic image capturing apparatus 1 equals the possible number of times of radiation emission N45 from the medical cart 40 (or the radiation generating apparatus 45) results in a maximum number of imaging operations with limited electric energy. Charge performed such that a difference between the number of capturable images N1 and the possible number of times of radiation emission N45 is small, if not identical, results in an increased number of imaging operations with limited power.

If the post-discharge electric energy E60 belongs to the second region R2 and the control device 58 of this embodiment determines charge to be available, the control device 58 calculates the possible number of times of radiation emission N45 based on the electric energy E60 remaining in the built-in power supply 60 in the medical cart 40 and the number of capturable images N1 based on the electric energy E24 remaining in the built-in power supply 24 in the radiographic image capturing apparatus 1. The control device 58 controls power fed from the built-in power supply 60 in the medical cart 40 to the built-in power supply 24 in the radiographic image capturing apparatus 1 such that the possible number of times of radiation emission N45 equals the number of capturable images N1 or a difference ΔN between them is reduced.

The electric energy required for the radiographic image capturing apparatus 1 to read signal values D for a single radiographic image is different from the electric energy required for the radiation generating apparatus 45 to emit radiation once. The control device 58 performs an appropriate conversion between them to calculate electric energy fed from the built-in power supply 60 in the medical cart 40 to the built-in power supply 24 in the radiographic image capturing apparatus 1.

This configuration can increase the number of capturable radiographic images N1 and thus the number of imaging operations to the maximum when imaging must be performed urgently with limited electric energy E60 remaining in the built-in power supply 60 in the medical cart 40 and limited electric energy E24 remaining in the built-in power supply 24 in the radiographic image capturing apparatus 1.

### Advantageous Effects

As shown above, the control device 58 in the radiographic image capturing system 100 according to this embodiment comprehensively manages the electric energy E60 remaining in the built-in power supply 60 in the medical cart 40 and the electric energy E24 remaining in the built-in power supply 24 in the radiographic image capturing apparatus 1 to determine the availability of charge of the built-in power supply 24 in the radiographic image capturing apparatus 1 with power from the built-in power supply 60 in the medical cart 40 based on the electric energy E60 remaining in the built-in power supply 60 in the medical cart 40. If the charge is determined to be available, charge is conducted; otherwise, no charge is conducted.

The control device 58 according to this embodiment, which comprehensively manage the electric energy E60 remaining in the built-in power supply 60 in the medical cart 40 and the electric energy E24 remaining in the built-in power supply 24 in the radiographic image capturing apparatus 1 can charge the built-in power supply 60 in the medical cart 40, without charging the built-in power supply 24, when the electric energy E60 remaining in the built-in power supply 60 in the medical cart 40 is not enough to charge the built-in power supply 24 in the radiographic image capturing apparatus 1.

The control device 58 switches the charge control properly in accordance with the electric energy E60 remaining in the built-in power supply 60 in the medical cart 40 and the electric energy E24 remaining in the built-in power supply 24 in the radiographic image capturing apparatus 1 to charge the built-in power supply 24 in the radiographic image capturing apparatus 1 properly with power from the built-in power supply 60 in the medical cart 40.

The configuration to manage only the electric energy E60 remaining in the built-in power supply 60 in the medical cart 40 or only the electric energy E24 remaining in the built-in power supply 24 in the radiographic image capturing apparatus 1 cannot precisely predict the electric energy E60 remaining in the built-in power supply 60 in the medical cart 40 when the built-in power supply 24 in the radiographic image capturing apparatus 1 is charged with power from the built-in power supply 60 in the medical cart 40, which may preclude an efficient operation of the radiographic image capturing system. In contrast, the radiographic image capturing system 100 according to this embodiment can avoid such a situation properly as the control device 58 comprehensively manages the remaining power as shown above.

The radiographic image capturing system 100 according to this embodiment can avoid the following risk, for example: The move of the medical cart 40 to a sickroom SR with an inadequate electric energy E60 remaining in the built-in power supply 60 in the medical cart 40 results in a transfer of the car to the charging station without capturing any image and then re-transfer to the sickroom SR for imaging. In this case, the radiographic image capturing system 100 prohibits imaging to allow the operator to move the medical cart 40 to the sickroom SR after charging it. This enables a smooth imaging and an efficient operation of the radiographic image capturing system 100.

The control device 58 according to the embodiment determines the number of imaging operations (i.e., the number of radiation emission operations from the radiation generating apparatus 45 or the number of read operations of the signal values D in the radiographic image capturing apparatus 1) based on the imaging order information, which is also applicable to the following embodiments.

However, in the case of emergency imaging, for example, the imaging order information may be created after imaging. The control device 58 may be informed of the number of imaging operations by any method other than the imaging order information, for example, an entry by the operator A.

### Second Embodiment

As described above, the control device 58 cannot automatically charge the built-in power supply 24 in the radiographic image capturing apparatus 1 with power from the built-in power supply 60 in the medical cart 40 when the connector 27 of the radiographic image capturing apparatus 1 is disconnected from the connector C at the end of the cable CA of the medical cart 40, i.e., after the radiographic image capturing apparatus 1 is removed from the cassette holder 48 of the medical cart 40 and the connector C is removed. The second embodiment will focuses on how the control device 58 controls charge during disconnection of the connector.

The control device 58 according to this embodiment obtains information on the voltage V24 of the built-in power supply 24 from the radiographic image capturing apparatus 1 through, for example, a wireless network and then calculates the electric energy E24 remaining in the built-in power supply 24 in the radiographic image capturing apparatus 1 based on the voltage information. The control device 58 determines the availability of charge of the built-in power supply 24 in the radiographic image capturing apparatus 1 based on the electric energy E24 and the electric energy E60 remaining in the built-in power supply 60 in the medical cart 40, similar to the first embodiment.

In this case, the control device 58 may retrieve the information on the voltage V24 of the built-in power supply 24 from the radiographic image capturing apparatus 1 on a regular basis or upon receiving a request for charging from the radiographic image capturing apparatus 1. Alternatively, the control device 58 may retrieve the voltage information when imaging order information is changed or added after disconnection of the connector 27 of the radiographic image capturing apparatus 1 from the connector C at the end of the cable CA of the medical cart 40.

However, the control device 58 according to this embodiment cannot automatically charge the built-in power supply 24 in the radiographic image capturing apparatus 1 because the connector 27 of the radiographic image capturing apparatus 1 is not connected to the connector C at the end of the cable CA of the medical cart 40. The control device 58 according to this embodiment displays a message on, for example, the display screen 51 of the medical cart 40 (see FIG. 4), makes a sound, lights or blinks the indicator 28 of the radiographic image capturing apparatus 1 (see FIG. 1) in a predetermined manner or color, or beeps to notify the operator A, such as a radiological technician, of the necessity for charge of the built-in power supply 24 in the radiographic image capturing apparatus 1 when the built-in power supply 24 in the radiographic image capturing apparatus 1 is charged with power from the built-in power supply 60 in the medical cart 40.

When the connector 27 of the radiographic image capturing apparatus 1 is connected to the connector C of the medical cart 40, the control device 58 controls the charging unit 62 of the medical cart 40 (see FIG. 6) to charge the built-in power supply 24 in the radiographic image capturing apparatus 1 with power from the built-in power supply 60 in the medical cart 40.

It is a common practice for the radiographic image capturing apparatus 1 to light or blink the indicator 28 in a predetermined manner or color or beep or for a console that has received a notice from the radiographic image capturing apparatus 1 to send a message on a display screen (not shown) or make a sound to notify the operator A of the necessity for charge of the radiographic image capturing apparatus 1 when the electric energy E24 remaining in the built-in power supply 24 in the radiographic image capturing apparatus 1 (or voltage V24) is reduced to a predetermined value.

In this embodiment, if the control device 58 determines to perform charge as described above, the control device 58 notifies the operator of the necessity for charge of the built-in power supply 24 in the radiographic image capturing apparatus 1 even if the electric energy E24 remaining in the built-in power supply 24 in the radiographic image capturing apparatus 1 (or the voltage V24) is still above the predetermined level which is provided to notify the operator of the necessity for charge. Such a notification is different from the usual one.

This configuration allows the control device 58 to instruct the operator A to connect the connector 27 of the radiographic image capturing apparatus 1 to the connector C at the end of the cable CA of the medical cart 40 to charge the built-in power supply 24 in the radiographic image capturing apparatus 1 with power from the built-in power supply 60 in the medical cart 40 properly when the built-in power supply 24 in the radiographic image capturing apparatus 1 needs to be charged.

Similar to the first embodiment, the control device 58 according to this embodiment comprehensively manages the electric energy E60 remaining in the built-in power supply 60 in the medical cart 40 and the electric energy E24 remaining in the built-in power supply 24 in the radiographic image capturing apparatus 1 and determines the availability of charge of the built-in power supply 24 in the radiographic image capturing apparatus 1 with power from the built-in power supply 60 in the medical cart 40 based on the electric energy E60 remaining in the built-in power supply 60 in the medical cart 40. If the charge is determined to be available, charge is conducted; otherwise, no charge is conducted. The radiographic image capturing system according to the second embodiment can provide the same advantageous effects as those of the radiographic image capturing system according to the first embodiment.

Different variations described in the first embodiment are also applicable to this embodiment. In particular, the control device 58 may direct the signal values D to be transferred from the radiographic image capturing apparatus 1 through a wireless network for each imaging operation or stored in the storage unit 23 without being transferred.

For example, when new imaging order information is added after the connector 27 of the radiographic image capturing apparatus 1 is disconnected from the connector C at the end of the cable CA of the medical cart 40, the control device 58 obtains information on the voltage V24 of the built-in power supply 24 from the radiographic image capturing apparatus 1 and calculates the electric energy E24 remaining in the built-in power supply 24 based on the obtained voltage V24.

Based on the calculated electric energy, the control device 58 direct the signal values D to be transferred from the radiographic image capturing apparatus 1 through a wireless network for each imaging or stored in the storage unit 23 without being transferred. This configuration is useful to prevent the following risk, for example: Transfer of the signal values D for each imaging in response to the addition of imaging order information results in the depletion of the electric energy E24 remaining in the built-in power supply 24 in the radiographic image capturing apparatus 1 (running out of battery) before all the imaging operations designated in the imaging order information, including newly added imaging order information, are completed. Accordingly, this configuration can ensure a proper imaging.

As described above, running out of battery may result in re-imaging. Advantageously, the above configuration can successfully avoid such a risk and allows the read signal values D to be stored in the storage unit 23 of the radiographic image capturing apparatus 1 properly without being transferred, thus enabling an efficient imaging.

### Third Embodiment

If the capacity of the built-in power supply 24 in the radiographic image capturing apparatus 1 increases in future as described above, charge could be applied to not only the built-in power supply 24 in the radiographic image capturing apparatus 1 with power from the built-in power supply 60 in the medical cart 40 just as in the first and second embodiments, but also the built-in power supply 60 in the medical cart 40 with power from the built-in power supply 24 in the radiographic image capturing apparatus 1.

The control device 58 can control the charge of the built-in power supply 24 in the radiographic image capturing apparatus 1 with power from the built-in power supply 60 in the medical cart 40 in the same manner as that in the first and second embodiments.

While the built-in power supply 60 in the medical cart 40 is charged with power from the built-in power supply 24 in the radiographic image capturing apparatus 1, the control device 58 manages the electric energy E24 remaining in the built-in power supply 24 in the radiographic image capturing apparatus 1 and determines, for example, the first region R1, the second region R2 and the third region R3 (not shown) for the electric energy E24 remaining in the built-in power supply 24 in the radiographic image capturing apparatus 1, like the electric energy E60 remaining in the built-in power supply 60 in the medical cart 40 shown in FIG. 8.

The control device 58 determines the availability of charge of the built-in power supply 60 in the medical cart 40 with power from the built-in power supply 24 in the radiographic image capturing apparatus 1 based on the electric energy E24 remaining in the built-in power supply 24 in the radiographic image capturing apparatus 1. If the charge is determined to be available, charge is conducted; otherwise, no charge is conducted.

This configuration can provide the same advantageous effects as those of the first and second embodiments and allows the built-in power supply 60 in the medical cart 40 to be charged properly with power from the built-in power supply 24 in the radiographic image capturing apparatus 1 if such charge is available.

If the electric energy E24 remaining in the built-in power supply 24 in the radiographic image capturing apparatus 1 is too low to charge, this configuration allows the control device 58 to prompt the operator A to charge the built-in power supply 24 in the radiographic image capturing apparatus 1, without performing the charge, enabling an efficient operation of the radiographic image capturing system 100, just as in the first and second embodiments.

In the first to third embodiments, the computer unit 58 in the medical cart 40 works as the control device. Alternatively, any other general computer, such as a console (not shown) mounted in the medical cart 40, for example, may be used as a control device. Alternatively, such a control device may be a dedicated device.

Alternatively, the control unit 22 (see FIG. 2) in the radiographic image capturing apparatus 1 may function as a control device. In this case, the control unit 22 in the radiographic image capturing apparatus 1 obtains information on the voltage V60 of the built-in power supply 60 in the medical cart 40 from, for example, the charging unit 62 (see FIG. 6), which functions as a voltage measuring unit for measuring the voltage V60 of the built-in power supply 60 in the medical cart 40. The control unit 22 calculates the electric energy E60 remaining in the built-in power supply 60 in the medical cart 40 based on the obtained information and controls charge as shown above based on the calculated electric energy E60 remaining in the built-in power supply 60 in the medical cart 40.

Alternatively, the medical cart 40 and the radiographic image capturing apparatus 1 each may have a control device and these control devices may control charge in a collaborative manner through exchange of signals and information between them.

The present invention should not be limited to simple-image or moving-image capturing and also applicable to tomosynthetic imaging, which involves multiple radiation emission operations to, for example, the subject H, and dual energy subtraction imaging.

### [Method for Charging Built-in Power Supply at Charging Station]

If the built-in power supply 24 in the radiographic image capturing apparatus 1 is not charged with power, for example, from the built-in power supply 60 in the medical cart 40 in the above embodiments, the medical cart 40 and/or the radiographic image capturing apparatus 1 are moved to the charging station and connected to an outlet for charge.

If the built-in power supply 60 in the medical cart 40 has a larger capacity than the built-in power supply 24 in the radiographic image capturing apparatus 1 like, for example, the first and second embodiments, the medical cart 40 may be charged prior to the radiographic image capturing apparatus 1. When the built-in power supply 60 in the medical cart 40 is previously charged, the built-in power supply 24 in the radiographic image capturing apparatus 1 can be charged properly with power from the built-in power supply 60, which has a larger capacity, in the medical cart 40 even if there is no enough time to charge the built-in power supply 24 in the radiographic image capturing apparatus 1.

If both of the built-in power supply 60 in the medical cart 40 and the built-in power supply 24 in the radiographic image capturing apparatus 1 have a large capacity, like, for example, the third embodiment, the built-in power supply 24 in the radiographic image capturing apparatus 1 or the built-in power supply 60 in the medical cart 40, whichever has a higher charging rate, may be charged first. This configuration allows the built-in power supply having a higher charging rate to be charged first and then the other built-in power supply to be charged promptly, resulting in a higher charging efficiency.

If imaging must be performed urgently before charge of the other built-in power supplies is completed, electric energy can be shared by feeding it from a built-in power supply charged first to the other built-in power supply. If the built-in power supply charged first does not have enough remaining electric energy, charge should be performed such that the possible number of times of radiation emission N45 from the radiation generating apparatus 45 in the medical cart 40 equals the number of capturable images N1 or a difference ΔN between them is small. This can maximize the number of imaging operations with limited electric energy.

The above embodiments have been described, but the embodiments should not be construed to limit the present invention, and various modifications may be made without departing from the scope of the invention.

The present U.S. patent application claims a priority under the Paris Convention of Japanese patent application No. 2016-018561 filed on Feb. 3, 2016, in which all contents of this application are disclosed, and which shall be a basis of correction of an incorrect translation.

What is claimed is:

1. A radiographic image capturing system comprising:
a medical cart which includes a radiation generating apparatus mounted on the medical cart, the radiation generating apparatus emitting radiation, and a first chargeable built-in power supply;
a portable radiographic image capturing apparatus which includes a plurality of radiation detecting elements two-dimensionally arranged and a second chargeable built-in power supply; and
a control device which manages electric energy remaining in the first built-in power supply in the medical cart and electric energy remaining in the second built-in power supply in the portable radiographic image capturing apparatus, determines availability of charge of the second built-in power supply in the portable radiographic image capturing apparatus with power from the first built-in power supply in the medical cart based on the electric energy remaining in the first built-in power supply, and performs control so as to allow the charge when the charge is determined to be available, and so as not to conduct the charge when the charge is determined to be unavailable.

2. The radiographic image capturing system of claim 1, wherein when the charge is determined to be available, the control device calculates a possible number of times of radiation emission from the radiation generating apparatus based on the electric energy remaining in the first built-in power supply in the medical cart, calculates a number of a radiographic image capable of being captured in the portable radiographic image capturing apparatus based on the electric energy remaining in the second built-in power supply in the portable radiographic image capturing apparatus, and controls the charge of the second built-in power supply in the portable radiographic image capturing apparatus with the power from the first built-in power supply in the medical cart such that the possible number of times of radiation emission is equal to the number of the radiographic image capable of being captured or a difference between the numbers is small.

3. The radiographic image capturing system of claim 1, wherein when the charge of the second built-in power supply in the portable radiographic image capturing apparatus with the power from the first built-in power supply in the medical cart reduces the electric energy remaining in the first built-in power supply in the medical cart to a level equal to or less than a sum of a lower limit of the electric energy in the first built-in power supply in the medical cart and electric energy required to move the medical cart or to a level equal to or less than a total of predetermined electric energy and the sum, the control device determines that the charge is unavailable and does not conduct the charge.

4. The radiographic image capturing system of claim 1, wherein when conducting the charge of the second built-in power supply in the portable radiographic image capturing apparatus with the power from the first built-in power supply in the medical cart, the control device automatically starts the charge if the first built-in power supply in the medical cart is electrically connected to the second built-in power supply in the portable radiographic image capturing apparatus.

5. The radiographic image capturing system of claim 1, wherein when conducting the charge of the second built-in power supply in the portable radiographic image capturing apparatus with the power from the first built-in power supply in the medical cart, the control device notifies an operator of necessity for charge of the second built-in power supply in the portable radiographic image capturing apparatus if the first built-in power supply in the medical cart is electrically disconnected to the second built-in power supply in the portable radiographic image capturing apparatus.

6. The radiographic image capturing system of claim 1, wherein when not conducting the charge of the second built-in power supply in the portable radiographic image capturing apparatus with the power from the first built-in power supply in the medical cart, the control device notifies an operator of unavailability of imaging and/or necessity for charge of the first built-in power supply in the medical cart.

7. The radiographic image capturing system of claim 1, wherein
the portable radiographic image capturing apparatus includes a wireless communication unit, and a storage unit which stores a signal value read in each of the radiation detecting elements,
when the electric energy remaining in the second built-in power supply in the portable radiographic image capturing apparatus is equal to or greater than electric energy required for imaging in the portable radiographic image capturing apparatus,
if the electric energy remaining in the second built-in power supply in the portable radiographic image capturing apparatus is enough to also transfer the read signal value to the outside of the portable radiographic image capturing apparatus via the wireless communication unit for each imaging, the control device instructs the portable radiographic image capturing apparatus to transfer the signal value via the wireless communication unit for each imaging, and
if the electric energy remaining in the second built-in power supply in the portable radiographic image capturing apparatus is not enough to also transfer the read signal value to the outside of the portable radiographic image capturing apparatus via the wireless communication unit for each imaging, the control device instructs the portable radiographic image capturing apparatus to store the signal value in the storage unit.

8. The radiographic image capturing system of claim 1, further comprising:
a voltage measuring unit which measures a voltage of the second built-in power supply in the portable radiographic image capturing apparatus, wherein
the control device manages the electric energy remaining in the first built-in power supply in the medical cart, and determines the availability of the charge of the second built-in power supply in the portable radiographic image capturing apparatus with the power from the first built-in power supply in the medical cart based on the electric energy remaining in the first built-in power supply in the medical cart when the electric energy remaining in the second built-in power supply in the portable radiographic image capturing apparatus is reduced to a level equal to or less than a predetermined electric energy, the electric energy remaining in the second built-in power supply being calculated based on the voltage of the second built-in power supply in the portable radiographic image capturing apparatus measured by the voltage measuring unit.

9. The radiographic image capturing system of claim 1, further comprising:
a voltage measuring unit which measures a voltage of the first built-in power supply in the medical cart, wherein
the control device manages the electric energy remaining in the second built-in power supply in the portable radiographic image capturing apparatus, calculates the electric energy remaining in the first built-in power supply in the medical cart based on the voltage of the first built-in power supply in the medical cart measured by the voltage measuring unit when the electric energy remaining in the second built-in power supply in the portable radiographic image capturing apparatus is reduced to a level equal to or less than a predetermined electric energy, and determines the availability of the charge of the second built-in power supply in the portable radiographic image capturing apparatus with the power from the first built-in power supply in the medical cart based on the calculated electric energy remaining in the first built-in power supply in the medical cart.

10. A radiographic image capturing system comprising:
a medical cart which includes a radiation generating apparatus mounted on the medical cart, the radiation generating apparatus emitting radiation, and a first chargeable built-in power supply;
a portable radiographic image capturing apparatus which includes a plurality of radiation detecting elements two-dimensionally arranged and a second chargeable built-in power supply; and
a control device which manages electric energy remaining in the first built-in power supply in the medical cart and electric energy remaining in the second built-in power supply in the portable radiographic image capturing apparatus, determines availability of charge of the second built-in power supply in the portable radiographic image capturing apparatus with power from the first built-in power supply in the medical cart or availability of charge of the first built-in power supply in the medical cart with power from the second built-in power supply in the portable radiographic image capturing apparatus based on the electric energy remaining in the first built-in power supply in the medical cart and the electric energy remaining in the second built-in power supply in the portable radiographic image capturing apparatus, and performs control so as to conduct the former charge when the former charge is determined to be available or so as to conduct the latter charge when the latter charge is determined to be available, and so as not to conduct the former charge when the former charge is determined to be unavailable or so as not to conduct the latter charge when the latter charge is determined to be unavailable.
